(19) United States
(12) Patent Application Publication
(10) Pub. No.: US 2026/0085271 A1
(43) Pub. Date: Mar. 26, 2026

(54) METHOD FOR OPERATING A BIOPROCESS INSTALLATION FOR PRODUCTION OF A BIOPRODUCT

(71) Applicant: Sartorius Stedim Biotech GmbH, Göettingen (DE)

(72) Inventors: Lukas Nik Reger, Goettingen (DE); Martin Saballus, Neu-Eichenberg (DE); Markus Kampmann, Dortmund (DE); Julia Niemann, Hannover (DE)

(21) Appl. No.: 19/111,657

(22) PCT Filed: Sep. 12, 2023
(86) PCT No.: PCT/EP2023/074979

§ 371 Date: Mar. 13, 2025

(30) Foreign Application Priority Data
Sep. 13, 2022 (EP) 22 195 394.6

(51) Int. Cl. C12M 1/36; C12M 1/00; C12M 1/34

(52) U.S. Cl. CPC C12M 41/48; C12M 29/00; C12M 41/12; C12M 41/26; C12M 41/32; C12M 41/34; C12M 41/36; C12M 41/40; C12M 41/42; C12M 47/10

(57) ABSTRACT

Method for operating a bioprocess installation for production of a bioproduct, wherein the bioprocess installation comprises a source receptacle for cell cultivation, a harvest receptacle for bioproduction and a clarification setup with a centrifuge wherein the source receptacle is operated in a cyclical production mode comprising the steps of: a) starting the cyclical production mode, b) cultivating the cells thereby obtaining a cell broth comprising cultivated cells, c) discharging a discharge fraction of the cell broth, d) combining a restart fraction of the cell broth with fresh cultivation medium and repeating step b), e) repeating steps c) and d) at least once and/or f) discharging the cell broth obtained from step d), obtaining a discharge fraction, wherein the method further comprises the steps: i) centrifuging the discharge fraction via the centrifuge, ii) operating the harvest receptacle in a production mode, wherein steps i) and ii) are executed at least twice.

### CROSS-REFERENCE TO RELATED APPLICATIONS

This application is a national stage application under 35 U.S.C. 371 of International Patent Application Serial No. PCT/EP2023/074979, entitled “METHOD FOR OPERATING A BIOPROCESS INSTALLATION FOR PRODUCTION OF A BIOPRODUCT,” filed Sep. 12, 2023, which claims priority from European Patent Application No. 22 195 394.6, filed Sep. 13, 2022, the disclosure of which is incorporated herein by reference.

### FIELD OF THE TECHNOLOGY

Various embodiments relate to a method for operating a bioprocess installation for production of a bioproduct.

### SUMMARY

The term “bioprocess” presently represents any kind of biotechnological processes, in particular biopharmaceutical processes. Hence, the proposed method may be applied in various fields of biotechnology and for different kinds of bioprocesses. An example of a bioprocess is the use of a bioreactor to cultivate microorganisms like bacterial or mammalian cells under given conditions to produce a bioproduct. Typically, a cell broth is transferred from the bioreactor to a downstream process to separate the bioproduct from the cells and to purify the bioproduct.

The term “bioproduct” in general refers to a product produced by the cells cultivated. Cultivation of cells may currently be used for the production of biopharmaceuticals, in particular proteins, such as human insulin, growth factors, hormones or vaccine proteins, in particular antibodies, antibody derivates, exosomes or the like. The bioproduct may as well be from a group of non-biopharmaceuticals, such as enzymes for food processing, laundry detergent enzymes, biodegradable plastics or biofuels. Additionally or alternatively, cells may also be cultured to produce viral vectors, including lentiviral vectors or the like that are purified and used for applications in the emerging field of cell and gene therapy.

The focus of various embodiments is on biopharmaceuticals, more particularly antibodies, and viral vectors that are secreted by the cells into the culture medium.

The term “cells” can represent mammalian cells, including HEK293 cells, CHO cells or the like. The cells can have been genetically modified, such as by transfection, to produce the desired bioproduct. In various embodiments, the bioproduct is secreted from the cells into the culture medium. Alternatively, the bioproduct stays within the cells.

The method in question for operating a bioprocess installation for production of a bioproduct may be applied in various fields of biotechnology and for different kinds of bioprocesses. Exemplarily, the method may be applied to produce a monoclonal antibody using a mammalian CHO suspension cell line.

When operating a bioprocess installation for the production of a bioproduct, process efficiency is particularly important. In particular, process efficiency relates to the cost-efficiency and the footprint of the process. High cost-efficiency and increasing flexibility are driven by the increasing demand for biopharmaceutical drugs, including monoclonal antibodies, vaccines and the like. Another reason for a cost-effective process design is that the material costs associated with the mentioned type of bioprocesses are typically high. A reason for this is that the sterility during the process has to be kept. As a consequence, the bioprocesses typically involve the use of single-use materials and/or cost-intensive sterilization procedures like heat sterilization or the like.

Cost-efficiency is not only related to material costs, but also to an efficient process design. Typically, to get a receptacle, in particular a bioreactor, to a state where it produces the bioproduct, a seed train involving multiple cultivation steps at increasing cultivation volumes needs to be performed. This seed train cultivation takes substantial time, because the cells need to be grown to a certain cell density in each step of the seed train to inoculate the next step and cell growth rates might be comparably low. Additionally, and depending on the final production scale, a substantial number of seed train bioreactors may be required that each need to be run consecutively. Consequently, there is a need to increase process efficiency. Further, it is desirable that the equipment utilization be as high as possible, meaning that especially the receptacles involved in the method are utilized to a high degree. Additionally, the process design in terms of equipment needed should be as simple as possible to keep the complexity as low as possible.

Document WO 99/33955 A1 relates to the cultivation of preferably anchorage-dependent cells and discloses a method for operating a bioprocess installation for production of a bioproduct, wherein the bioprocess installation comprises a source receptacle for cell cultivation and a harvest receptacle for bioproduction. It has been realized that a fraction of the cells from the source receptacle might be reused as a restart fraction while another fraction of the cells is used for bioproduction. However, there is still a need to improve the production efficiency.

Document WO 2020/010080 A1 relates to a method for culturing mammalian cells in a source receptacle and strongly increasing the cell concentration by adjusting the cultivation medium before transferring it into a harvest receptacle. This is used to achieve cell densities as in perfusion culture without using perfusion culture.

It is a challenge to improve on the known prior art to achieve higher process efficiency.

Various embodiments are based on the problem of improving the known method such that a further optimization regarding the named challenge is reached.

The above-noted object is solved by various features provided herein.

The main realization of some embodiments is that operating a source receptacle in a cyclical production mode to produce cells and operating a harvest receptacle in a non-cyclical production to produce a bioproduct enables an efficient supply of cells to the harvest receptacle and allows a separate optimization of both process steps, including process parameters like initial viable cell concentration and culture medium chosen.

The source receptacle is used to cultivate cells and a fraction of those cells is transferred to a harvest receptacle, where the cells mainly produce bioproduct. Between these steps the cells are centrifuged, allowing to harvest the bioproduct produced in the source receptacle as a collateral, further allowing the increase of the viable cell concentration, allowing to remove potentially toxic byproducts and allowing to change the process conditions between the receptacles. In particular, there may be different optimal process conditions for the production of cells and bioproduct.

By using separate receptacles, the cell production in the source receptacle is particularly simple, while a non-cyclical operation of the harvest receptacle enables adjusting the process conditions in the harvest receptacle to favor bioproduction without having to keep the cells viable. Further, by using the first receptacle for repeated cell production, time, labour-and cost-intensive seed train cultivation may be greatly reduced.

By including a clarification setup with a centrifuge, the process conditions between the source and the harvest receptacle may be adjusted in a particularly simple manner as the cells may easily be washed before being transferred to the harvest receptacle. This way, dead cells and cell debris may also be at least partially removed from the cell broth, which results in an increase in culture viability. Further, washing enables potentially cell-growth inhibiting compounds, e.g., byproducts like lactate and/or ammonium, to be removed or at least reduced from the culture medium. This may enhance the subsequent cultivation in the harvest receptacle and/or simplify the further purification of the bioproduct.

Further, utilization of a centrifuge enables an easy separation of the bioproduct and the cells so that the bioproduct may also be recovered during transfer from the source receptacle to the harvest receptacle.

By operating the source receptacle in a cyclical production mode and the harvest receptacle in a non-cyclical production mode, the process costs and the footprint may be reduced as no large media storage is required and no means for cell retention need to be provided by either receptacle, in particular compared to a perfusion-based process. The utilization of two separate receptacles further provides the option to adapt the cultivation times in each receptacle to each other so that the equipment utilization rate is increased. This further enhances the process efficiency. By using a restart fraction of cells to start the next cultivation in the source receptacle, the downtime of the source receptacle is largely reduced as the source receptacle might be used directly for the next production cycle and does not have to be cleaned.

Proposed is a method for operating a bioprocess installation for production of a bioproduct, wherein the bioprocess installation comprises a source receptacle for cell cultivation, a harvest receptacle for bioproduction and a clarification setup with a centrifuge, wherein the source receptacle is operated in a cyclical production mode, wherein the cyclical production mode comprises, in this order, the steps of:
a) starting the cyclical production mode in the source receptacle with initial cells and cultivation medium,
b) cultivating the cells in the source receptacle, thereby obtaining a cell broth comprising cultivated cells, whereby the source receptacle and the cultivation medium are configured to provide source culture environment conditions for the cultivation of the cells,
c) discharging a discharge fraction of the cell broth from the source receptacle,
d) combining a restart fraction of the cell broth with fresh cultivation medium and repeating step b),
e) repeating steps c) and d) at least once and/or
f) discharging the cell broth obtained from step d) from the source receptacle stopping the cyclical production mode, obtaining a discharge fraction,
wherein the method further comprises the steps of:
i) transferring the discharge fraction to the clarification setup and centrifuging the discharge fraction via the centrifuge, thereby separating the discharge fraction into at least a centrifuged discharge fraction, supernatant and in some embodiments bioproduct,
ii) transferring at least part of the centrifuged discharge fraction from step i) and fresh cultivation medium into the harvest receptacle and operating the harvest receptacle in a production mode, whereby the harvest receptacle and the cultivation medium are configured to provide harvest culture environment conditions, producing the bioproduct in the harvest receptacle,
wherein steps i) and ii) are executed at least twice with discharge fractions from an execution of step c) and/or step f).

The term “cell broth” means a suspension of solid cells and possibly cell debris in a cultivation medium and describes the entirety of the cultivation medium and the respective cells cultured in the cultivation medium.

According to various embodiments, the cultivation in the source receptacle and the cultivation in the harvest receptacle are carried out in parallel for a substantial amount of time. This way, the process efficiency is drastically enhanced as the equipment utilization is increased with the receptacles being utilized to a large extent.

Various embodiments are directed towards the synchronization between the cultivation time in the source receptacle and the production time in the harvest receptacle. Especially when the number of available receptacle slots is limited, a synchronization between the cultivation time and the production time is important to keep the process as efficient as possible. By synchronizing the cultivation times, extensive downtimes of either receptacle may be reduced, which increases the equipment utilization rate and therefore the process efficiency.

By repeating the cyclical production mode without breaking the synchronicity between the source receptacle slot and the harvest receptacle slot, the duration of the seed train is significantly shortened as not all steps of the seed train need to be repeated for each consecutive transfer of cells to the harvest receptacle. Further, each subsequent cyclical production in the source receptacle may be performed at the same source receptacle slot, which reduces the overall footprint of the process as no additional receptacle slots are required. Again, this also increases the equipment utilization rate as not only the receptacles themselves, but also the infrastructure at the respective receptacle slot is utilized as far as possible.

According to various embodiments, the harvest fraction from the harvest receptacle is subjected to the clarification setup after producing the bioproduct. Here, the use of a centrifuge in the clarification setup is particularly advantageous as it allows the discharging from the source and harvest receptacles to be carried out process- and equipment-wise similarly, leading to easier work steps and by using the same centrifuge also leading to higher equipment usage time.

Using a sensor arrangement to measure at least one parameter of the source receptacle and/or the harvest receptacle enables to determine the remaining cultivation time and/or the remaining production time. This allows adapting some process step times, in particular the duration of step b), and thereby keeping the synchronization between the cultivation times in the source receptacle and in the harvest receptacle even when unexpected changes occur.

Providing a source electronic process control to control the source culture environment conditions as proposed in various embodiments can be advantageous as the source receptacle can be controlled to change its culturing conditions, cell growth rate and the like if necessary. For example, if the harvest receptacle is to be used for production for a longer than planned time, the source receptacle may be adapted to decrease the cell growth rate. Thereby, a target number of cells is reached later, synchronizing the source receptacle and the harvest receptacle instead of having to throw away part of the cell broth from the source receptacle if it reaches its target cell amount too early or instead of risking that the cells lose their viability and may not be used anymore. Further, if bioproduction already occurs in the source receptacle, the amount of bioproduct in the source receptacle might well be increased, as the adaptation of the source receptacle might result in starting and/or increasing bioproduction. Nevertheless, producing a target amount of cells and maintaining a certain viability of the cells remains the priority within the source receptacle.

By predicting the time when a termination condition is reached in the harvest receptacle and adjusting the cultivation conditions in the source receptacle based on this prediction, the process flexibility is largely increased as the production can be continued in the harvest receptacle and cell growth in the source receptacle can be adjusted accordingly. This way, the production time in the harvest receptacle can be changed without having to repeat the complete seed train. In particular, the source receptacle may not be optimized for maximum cell growth but instead still have some room to increase, and in some embodiments also decrease, cell growth, thereby providing a control reserve and producing more bioproduct in the source receptacle. Providing a control reserve is a particularly interesting possibility that the proposed method provides. In various embodiments, a harvest receptacle would usually be timed onto a seed train, not allowing greater deviations in the harvest receptacle runtime, even when the cells might still have a high viability and a good production rate. By being able to use the cells for a longer time without desynchronizing the seed train, waste and cost per product are reduced.

Various embodiments detail possible options to synchronize the source receptacle and the harvest receptacle. To reach a pre-defined cell quantity in the source receptacle needed for subsequent production of the bioproduct in the harvest receptacle, a synchronization strategy may be implemented.

By increasing the cell concentration between step b) and step ii), the subsequent production of the bioproduct in step ii) may be accelerated. Increasing the viable cell concentration by centrifugation is particularly advantageous, as not only the cells may be separated from the bioproduct already produced, but the cells may also be washed and/or transferred into another cultivation medium.

By changing at least one process parameter of the source culture environment conditions compared to the harvest culture environment conditions, cell cultivation and bioproduction can be efficiently split between the receptacles. In the harvest receptacle, the cell viability does not need to be kept above a certain threshold as the cells can be discharged at the end of the production. Using a centrifuge further makes it a lot easier to change the culture environment between cultivation of the cells and production of the bioproduct.

In various embodiments, the source culture environment and/or the harvest culture environment may be, at least initially, identical for multiple repetitions of steps b) and ii), making the overall method a repetition of identical parallel cultivation and production cycles.

By using batch and/or fed-batch cultivation conditions, the overall set-up is simplified, especially in comparison to perfusion-based systems as no means for retaining the cells within either receptacle during cultivation are required. This improves the cost-efficiency of the overall process and simplifies operating the bioprocess installation as process complexity is reduced. Further, no large storage space is required to continuously supply the needed amount of culture medium. In contrast to perfusion cultivation the contamination risk, the footprint in form of storage and waste of cultivation medium and the complexity are reduced.

In various embodiments, the properties of the centrifuge as well as the cell washing step and cell discharging step are specified. These specifications allow for an optimized preparation of the cells during forward or backward operation of the centrifuge, as well as for optimized conditions for the subsequent bioproduction. The specified centrifuge is particularly well suited for the proposed method.

By transfecting the cells after step c), bioproduction may only be enabled after the cells have been discharged from the source receptacle. This way, no bioproduction occurs in the source receptacle, which is a particularly efficient option to separate the bioproduction from cell growth.

The term “transfection” means that a transfer of genetic material is carried out to genetically modify the cells in such a way that the cells produce the desired bioproduct. Cell transfection may be carried out in different ways, including techniques such as electroporation, treatment with chemicals like calcium phosphate, microinjection or the like. In various embodiments, and as will be explained later, transfection is performed by treatment with chemicals. Alternatively, transfection may also be carried out using viruses or viral vectors.

Various embodiments provide a method for operating a bioprocess installation for production of a bioproduct, wherein the bioprocess installation comprises a source receptacle for cell cultivation, a harvest receptacle for bioproduction and a clarification setup with a centrifuge, wherein the source receptacle is operated in a cyclical production mode, wherein the cyclical production mode comprises, in this order, the steps of: a) starting the cyclical production mode in the source receptacle with initial cells and cultivation medium, b) cultivating the cells in the source receptacle, thereby obtaining a cell broth comprising cultivated cells, whereby the source receptacle and the cultivation medium are configured to provide source culture environment conditions for the cultivation of the cells, c) discharging a discharge fraction of the cell broth from the source receptacle, d) combining a restart fraction of the cell broth with fresh cultivation medium and repeating step b), e) repeating steps c) and d) at least once and/or f) discharging the cell broth obtained from step d) from the source receptacle stopping the cyclical production mode, obtaining a discharge fraction, wherein the method further comprises the steps of: i) transferring the discharge fraction to the clarification setup and centrifuging the discharge fraction via the centrifuge, thereby separating the discharge fraction into at least a centrifuged discharge fraction and supernatant and in some embodiments bioproduct, ii) transferring at least part of the centrifuged discharge fraction from step i) and fresh cultivation medium into the harvest receptacle and operating the harvest receptacle in a production mode, whereby the harvest receptacle and the cultivation medium are configured to provide harvest culture environment conditions, producing the bioproduct in the harvest receptacle, wherein steps i) and ii) are executed at least twice with discharge fractions from an execution of step c) and/or step f).

In various embodiments, for at least one, at least two, or each execution of steps b) and ii), the cultivation of cells in step b) and the bioproduction in step ii) are executed at least partially in parallel, such as, that the executions of steps b) and ii) run for at least 50%, at least 75%, or at least 90%, of the time used for cultivation in step b) and/or the time used for bioproduction in step ii) and/or the time used for execution of step b) and/or step ii) in parallel.

In various embodiments, a single source receptacle slot is paired with a single harvest receptacle slot, that step b) is executed for a, in particular pre-determined, cultivation time, that step ii) is executed for a, in particular pre-determined, production time, that the cultivation time and the production time and therefore the source receptacle slot and the harvest receptacle slot are synchronized such that for at least two consecutive executions, such as all executions, of steps ii) the same harvest receptacle slot is used.

In various embodiments, steps a) to f) are synchronized with repeated seed trains such that after step f) step a) is repeated at the same source receptacle slot without breaking synchronicity between the source receptacle slot and the harvest receptacle slot.

In various embodiments, the method further comprises the step of
iii) transferring a harvest fraction from the harvest receptacle to the clarification setup and centrifuging the harvest fraction via the centrifuge, thereby separating the bioproduct from the harvest fraction. In some embodiments, step iii) is executed after each step ii).

In various embodiments, the bioprocess installation comprises a sensor arrangement, that the sensor arrangement comprises at least one sensor placed in or at the source receptacle and/or at least one sensor placed in or at the harvest receptacle, in various embodiments, that at least one sensor is configured to measure at least one process parameter out of the group of carbon-source concentration, nitrogen-source concentration, amino acid concentration, growth factor concentration, oxygen concentration, carbon dioxide concentration, pH, temperature, conductivity, pressure, biomass concentration, biomass production rate, product concentration, productivity, oxygen uptake rate and/or stirring speed for the source receptacle and/or for the harvest receptacle.

In various embodiments, the bioprocess installation, in particular the source receptacle slot, comprises a source electronic process control for controlling the source culture environment conditions, such as that the source electronic process control is connected to the at least one sensor placed in or at the source receptacle.

In various embodiments, step ii) is terminated after a pre-determined termination condition is reached, in various embodiments, that at least one sensor measurement related to the termination condition is measured repeatedly, in particular cyclically or continuously, by the sensor placed in or at the harvest receptacle during at least one, in particular each, execution of step ii), in various embodiments, that the sensor measurement is used, in particular by a harvest electronic process control, to predict a time of reaching the termination condition, that the source culture environment conditions in step b) are controlled, in particular by the source electronic process control, to synchronize step b) with step ii).

In various embodiments, if a deviation between the predicted time of reaching the termination condition and a planned end of the production time is detected, in particular by the source electronic process control or the harvest electronic process control, a synchronization strategy is executed, in various embodiments, that the synchronization strategy comprises adapting the source culture environment conditions and/or harvest culture environment conditions to reduce the resulting deviation between the source receptacle and the harvest receptacle, in particular to reach a pre-defined absolute cell quantity in the source receptacle prior to initiating step c).

In various embodiments, an initial viable cell concentration in step ii) is higher than a final viable cell concentration in step b), in various embodiments, that the cell concentration is increased by the centrifugation in step i).

In various embodiments, at least one process parameter of the source culture environment conditions, in particular initial source culture environment conditions, in at least one, in particular each, execution of step b) and of the harvest culture environment conditions, in particular initial harvest culture environment conditions, in at least one, in particular each, execution of step ii) is set differently, in various embodiments, that the at least one process parameter is optimized in the source culture environment conditions for cultivation of cells over production of bioproduct and/or that the at least one process parameter is optimized in the harvest culture environment conditions for production of bioproduct over cultivation of cells.

In various embodiments, the initial source culture environment, in particular the source culture environment, and/or the initial harvest culture environment, in particular the harvest culture environment, is essentially identical for at least one, in particular each, repetition of step b) and/or step ii) respectively.

In various embodiments, cultivating the cells in step b) of the cyclical production mode is performed under batch cultivation conditions or fed-batch cultivation conditions, and/or, that the production mode of step ii) is performed under fed-batch cultivation conditions.

In various embodiments, the centrifuge is a fluidized bed centrifuge, in various embodiments, that the centrifuge is operated in a forward operation for cell separation and/or cell washing and a backward operation for cell discharging, in some embodiments, that the centrifuge is operated in a backward operation for transferring the centrifuged discharge fraction to the harvest receptacle between steps i) and ii).

In various embodiments, a transfection step is carried out after step c) on the discharge fraction, or, that the bioproduct is produced by the cells in the source receptacle.

### BRIEF DESCRIPTION OF THE DRAWINGS

In the following, various embodiments are explained with respect to the drawing. The drawing shows in

FIG. 1, a bioprocess installation to perform the proposed method for operating a bioprocess installation according to various embodiments,

FIG. 2, steps a)-e) of the proposed method for operating a bioprocess installation,

FIG. 3, steps e)-iii) of the proposed method for operating a bioprocess installation,

FIG. 4, an example for synchronizing cultivation in the source receptacle and the harvest receptacle, respectively,

FIG. 5, a source receptacle in a source receptacle slot, a harvest receptacle in a harvest receptacle slot and a clarification setup in an exemplary embodiment of the present application and

FIG. 6, proof-of-concept experiments.

### DETAILED DESCRIPTION

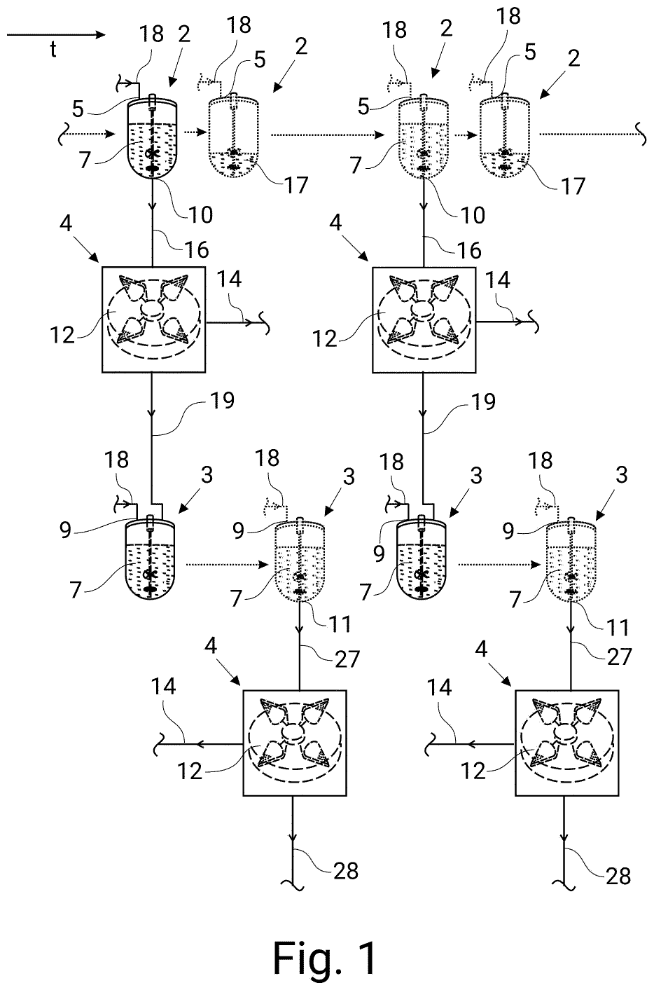

The proposed method for operating a bioprocess installation 1 is depicted in FIG. 1. FIG. 1 depicts two cycles of some embodiments of the method whereby FIG. 1 schematically shows the method over time from left to right. The bioprocess installation 1 comprises a source receptacle 2 for cell cultivation, a harvest receptacle 3 for bioproduction and at least one clarification setup 4.

In various embodiments and as depicted in FIG. 1, the source receptacle 2 is a bioreactor. It is further possible in some embodiments that a material of the source receptacle 2 is a single-use material. Alternatively, the source receptacle 2 may be a stainless-steel tank bioreactor. The source receptacle 2, in various embodiments, comprises at least one source receptacle inlet port 5 for the addition of liquids. Each source receptacle inlet port 5 may be connected to a respective liquid line providing the liquid.

The term “liquid” refers to any process liquid handled in the proposed bioprocess installation 1. This includes reagents added to control the source culture environment conditions and/or harvest culture environment conditions, e.g., acid or base to control the pH value, but also washing solutions like buffers used. In general, the term liquids may also refer to culture medium and/or feed medium 6 and/or the cell broth 7.

The term “cultivation medium” relates to the fact that the cells used for the bioprocess grow in or on specially designed solid, semi-solid or liquid cultivation medium, which supply the nutrients required by the respective organisms or cells. A variety of media exist, but usually contain at least a carbon source, a nitrogen source, water, salts, and micronutrients. Here, it can be that a chemically-defined cultivation medium 8 is used, meaning that all components of the medium and their respective concentrations are known. However, it is also well possible that a chemically-undefined medium is used, which may contain unknown nutrients and/or nutrients in unknown amounts. A typically example for a chemically undefined medium is a medium that contains FBS (fetal bovine serum).

As noted above, the bioprocess installation 1 further comprises a harvest receptacle 3. In various embodiments and as depicted in FIG. 1, the harvest receptacle 3 is a bioreactor. It is further possible that the material of the harvest receptacle 3 is a single-use material. Alternatively, the harvest receptacle 3 may be a stainless-steel tank bioreactor. In various embodiments, the harvest receptacle 3 further comprises at least one harvest receptacle inlet port 9 for the addition of liquids. Each harvest receptacle inlet port 9 may be connected to a respective liquid line providing the liquid.

In various embodiments and as depicted in FIG. 1, different harvest receptacles 3 are used throughout the method, which will be further explained below.

As depicted in FIG. 1, the source receptacle 2 and/or the harvest receptacle 3 can include at least one source receptacle outlet port 10 and/or at least one harvest receptacle outlet port 11. In various embodiments, the respective outlet ports are used to withdraw a liquid from the source receptacle 2 and/or the harvest receptacle 3. It can be that cell broth 7 is withdrawn from the source receptacle 2 and/or the harvest receptacle 3.

In various embodiments, the source receptacle 2 and/or the harvest receptacle 3 are designed to receive a liquid volume of more than 5 liters, more than 15 liters, or more than 50 liters. In various embodiments, the source receptacle 2 and the harvest receptacle 3 are designed to receive the same maximum liquid volume. It is further possible that the source receptacle 2 and the harvest receptacle 3 are of the same geometry. In various embodiments, also the layout and number of inlet ports and/or outlet ports is identical in the source receptacle 2 and in the harvest receptacle 3. This makes operation of both receptacles simpler.

However, it is also well possible that the source receptacle 2 and the harvest receptacle 3 are different in at least one or all aspects mentioned above. For example, the maximum liquid volume of the harvest receptacle 3 may be lower than the maximum liquid volume of the source receptacle 2.

As also mentioned above, the bioprocess installation 1 further comprises a clarification setup 4. In various embodiments, this clarification setup 4 carries out a physical process using centrifugal force to remove suspended solids, such as cells, from a liquid phase. In general, the proposed clarification setup 4 can be used to separate any solid/liquid components from each other, including but not limited to cells and media. In various embodiments, the clarification setup 4 comprises a centrifuge 12 for the clarification of the cell broth 7 by centrifugation. “Centrifugation” is a term for sedimentation of particles in an artificially, by centrifugal forces created, gravitational field, wherein a significant reduction of separation time is achieved via large accelerating forces.

For performing the proposed method, in various embodiments and also indicated in FIG. 1, that the source receptacle 2 and the centrifuge 12 may be brought into a fluidic coupling. It can be that the centrifuge 12 and the harvest receptacle 3 may be brought into a fluidic coupling as well.

The term “fluidic coupling” means that a fluidic connection is established that may be used to transfer a fluid, such as a liquid or at least part of the cell broth 7, from the source receptacle 2 to the centrifuge 12 and/or from the harvest receptacle 3 to the centrifuge 12 and/or to transfer a fluid, such as a liquid or the centrifuged 12 cell broth 7 from the centrifuge 12 to the harvest receptacle 3 and/or to a waste 13 location and/or to transfer the supernatant 14 containing the bioproduct from the centrifuge 12 to further purification steps 29.

The centrifuge 12 may be operated in a forward operation and/or in a backward operation and comprises a liquid network of interconnected liquid lines to establish different fluidic connections to and from the centrifuge 12.

In detail proposed is a method for operating a bioprocess installation 1 for production of a bioproduct, wherein the bioprocess installation 1 comprises a source receptacle 2 for cell cultivation, a harvest receptacle 3 for bioproduction and a clarification setup 4 with a centrifuge 12.

Proposed is, that the source receptacle 2 is operated in a cyclical production mode. The cyclical production mode comprises, in this order, the steps of:
a) starting the cyclical production mode in the source receptacle 2 with initial cells 15 and cultivation medium 8,
b) cultivating the cells in the source receptacle 2, thereby obtaining a cell broth 7 comprising cultivated cells, whereby the source receptacle 2 and the cultivation medium 8 are configured to provide source culture environment conditions for the cultivation of the cells,
c) discharging a discharge fraction 16 of the cell broth 7 from the source receptacle 2,
d) combining a restart fraction 17 of the cell broth 7 with fresh cultivation medium 8 and repeating step b),
e) repeating steps c) and d) at least once and/or
f) discharging the cell broth 7 obtained from step d) from the source receptacle 2 stopping the cyclical production mode, obtaining a discharge fraction 16.

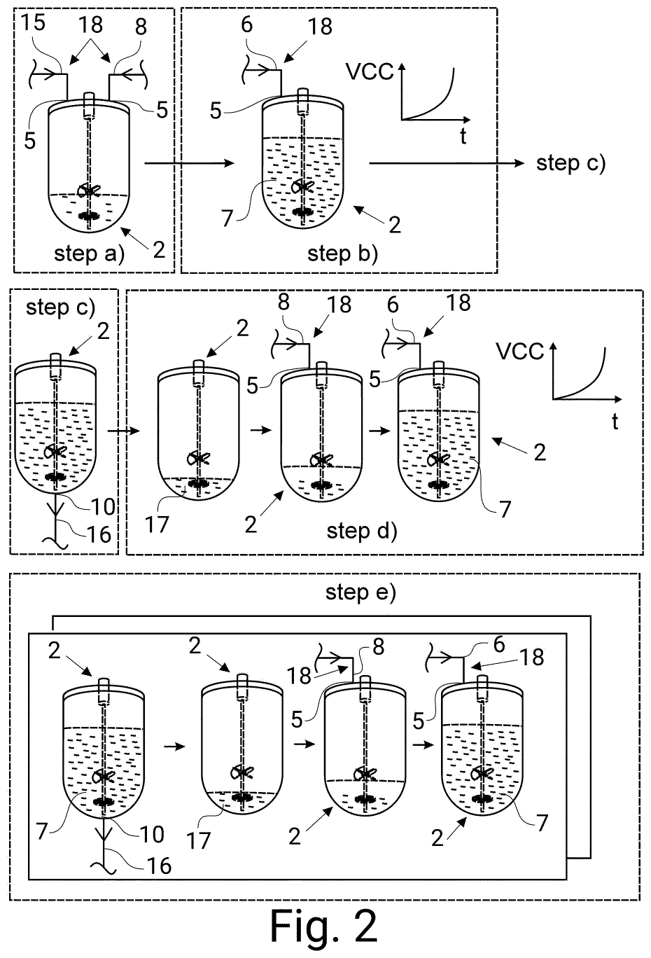

FIG. 1 shows most of the whole method and FIG. 2 shows steps a) to e) named respectively. Steps a) to f) relate to the source receptacle 2, the harvest receptacle 3 will be further explained below. The source receptacle 2 is run in a cyclical production mode to cultivate cells. In step a), the cultivation of cells starts by adding initial cells 15 into the source receptacle 2 as depicted in FIG. 2 on the top left. FIG. 2 shows how initial cells 15 and cultivation medium 8 are combined in the source receptacle 2 in the usual manner to cultivate the initial cells 15 and increase the absolute number of cells in the source receptacle 2. The initial cells 15 may be the result of a seed train which the cultivation in the source receptacle 2 may be the last step of. In various embodiments, the initial concentration of viable cells in the source receptacle 2 is adjusted to a predefined value. In various embodiments, this adjustment is performed by adding cultivation medium 8 until the predefined cell concentration is reached.

Step b) comprises the cultivation. The source receptacle 2 and the cultivation medium 8 together provide, in particular pre-defined, source culture environment conditions. Culture environment conditions comprise all relevant parameters for the cultivation of cells and the production of bioproduct like temperature, stirring speed, pH value, nutrient concentration, etc. The culture environment conditions, partially, change over time. Starting with initial culture environment conditions, the cells consume nutrients and secrete byproducts. Depending on the culture environment conditions and the cells, bioproduct may also be produced to some extent during cultivation in the source receptacle 2. This will be explained further below. In various embodiments the initial source culture environment conditions and/or yet to be explained initial harvest culture environment conditions are pre-defined, meaning there is a plan for the initial source and/or harvest culture environment conditions. In various embodiments, the later culture environment conditions and therefore all culture environment conditions, for the source and/or harvest receptacle 3, are pre-defined. However, that does not necessarily mean that every parameter is perfectly controlled. In various embodiments, the source culture environment conditions are adjusted to provide conditions that lead to an optimized cell growth and/or a high overall viability of the cells.

To maintain optimum cell growth conditions and to keep the viability of the cells above a predefined value, it may be required to feed a feed medium 6 during the cultivation of the cells in step b), because certain media components (e.g., a carbon source like glucose) may be depleted from the cultivation medium 8 during the cultivation as they are entirely consumed by the cells. However, depending on the culture behavior and the cultivation time, feeding cultivation medium 8 is only optional and may not be required in any case.

Here it should be noted that the feed medium 6 might be equal to the initial cultivation medium 8. Alternatively, and in various embodiments, the feed medium 6 differs in at least one type and/or at least one concentration of a component from the cultivation medium 8.

In various embodiments, at least one of the source receptacle inlet ports 5 is a feeding port with a feed line 18 for a controlled feeding of cultivation medium 8 during cultivation according to a predefined feeding profile. Such feeding profiles can be designed as pulse or continuous or a mixed feeding profile, wherein the feeding profile can be, in various embodiments, individually controllable and adjustable. It should be noted that feeding of the cultivation medium 8 results in an increase of the culture broth volume in the source receptacle 2 that depends on the feeding profile and the feed medium 6.

In various embodiments, after the cultivation of cells in step b) has reached a pre-determined termination condition, such as a target absolute number of cells or a target cell concentration, step b) is terminated and step c) is started. In various embodiments, step b) is focused on providing a fast increase in the number of cells while keeping the cells viable instead of producing bioproduct. Bioproduct may or may not be produced in step b). The bioproduct produced in step b), if any, may have been secreted into the cell broth 7 or may be located inside the cells. In the former case, the bioproduct may be harvested as will be explained later, in the latter case, the bioproduct is, in various embodiments, not harvested until after the cells have been used to produce bioproduct in the harvest receptacle 3 as will be explained.

In step c), a discharge fraction 16 of the cell broth 7 is removed from the source receptacle 2. This is shown in FIG. 1 moving downwards from the source receptacle 2. In various embodiments and as will be further detailed below, the cell broth 7 is not completely discharged from the source receptacle 2, but a restart fraction 17 is kept in the source receptacle 2. This restart fraction 17 is, in step d), combined with fresh cultivation medium 8 to again cultivate cells in a repetition of step b).

This, at least one, repetition of step b) makes the cultivation in the source receptacle 2 cyclical. Instead of using all cell broth 7 to produce bioproduct, the beginning of the next seed train is skipped, and the restart fraction 17 is used to again cultivate cells in the same source receptacle 2. In various embodiments, the restart fraction 17 stays in the source receptacle 2 and is not temporarily removed from the source receptacle 2 between repetitions of step b). That way the source receptacle 2 can be used for more than one cycle, without changing or cleaning the source receptacle 2. This is indicated by the dashed lines used for the source receptacle 2 in FIG. 1. The repetition of step b) is shown when moving at the top of FIG. 1 to the right and/or as part of step d) depicted in FIG. 2.

The repetition of step b), such as each repetition of step b), may be identical to the first execution of step b) in some or all aspects, in particular regarding the source environment conditions or some of the source environment conditions. All aspects described herein with regard to step b) may be identical for at least one, such as all executions of step b), alone or in any combination.

The restart fraction 17 and the discharge fraction 16 can sum up to at least 95%, at least 99%, or 100% of the cell broth 7 in the source receptacle 2 when starting step c) or ending step b).

In various embodiments, the discharge fraction 16 is at least 50%, at least 70%, or at least 90% of the volume of the cell broth 7 at the beginning of step c) or at the end of step b). In various embodiments, the cell broth 7 is discharged from the source receptacle 2 using the source receptacle outlet port 10. As will be explained in detail below, it can be that the cell broth 7 is transferred to the centrifuge 12.

In step e), steps c) and d) may be repeated for at least one, at least two, or at least three times. For each repetition, the discharge fraction 16 is transferred to a harvest receptacle 3 as will be explained. Therefore, the source receptacle 2 cyclically produces cell broth 7 for bioproduct production without needing a seed train each time.

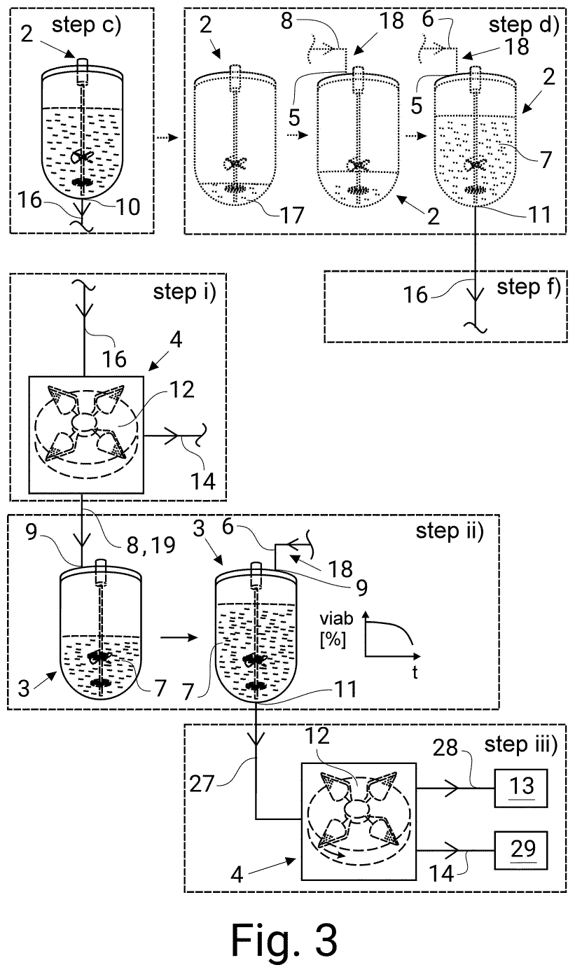

In step f), which is depicted in FIG. 3, the cyclical production is stopped. A discharge fraction 16 which can be at least 95%, at least 99%, or 100% of the cell broth 7 in the source receptacle 2, is, in various embodiments, again transferred to a harvest receptacle 3 as will be explained. FIG. 2 shows steps a) to e). FIG. 2 is to be read from left to right in each of the three rows. VCC is the viable cell concentration in the source receptacle 2 which increases and decreases with the different method steps. The volume in the source receptacle 2 may (as shown) or may not change during the steps, depending on the specifics of the process.

The discharge fraction 16, whether from step d) or step f), is not directly transferred to the harvest receptacle 3, but submitted to a clarification setup 4.

Turning now to the harvest receptacle 3 side and the transfer to the harvest receptacle 3 shown in FIG. 1 below the first row and in FIG. 3, the proposed method further comprises the steps of:
i) transferring the discharge fraction 16, resulting either from step c) or f), to the clarification setup 4 and centrifuging the discharge fraction 16 via the centrifuge 12, thereby separating the discharge fraction 16 into at least a centrifuged discharge fraction 19 and supernatant 14 and in various embodiments bioproduct,
ii) transferring at least part of the centrifuged discharge fraction 19 from step i) and fresh cultivation medium 8 into the harvest receptacle 3 and operating the harvest receptacle 3 in a production mode, in various embodiments a non-cyclical production mode, whereby the harvest receptacle 3 and the cultivation medium 8 are configured to provide harvest culture environment conditions, producing the bioproduct in the harvest receptacle 3, wherein steps i) and ii) are executed at least twice with discharge fractions 16 from an execution of step c) and/or step f).

Step i) relates to the centrifugation of the discharge fraction 16 via the centrifuge 12, in some embodiments always the same centrifuge 12. This centrifugation can be used to achieve several positive effects.

The discharge fraction 16 can be transferred to the centrifuge 12 via a multi-use or temporary liquid line. Multi-use means that the liquid line is used for at least two discharge fractions 16. A pumping arrangement may be used to transfer the discharge fraction 16. The discharge fraction 16 is then centrifuged 12. This centrifugation may increase the cell concentration such that a higher initial cell concentration may be used in step ii). The cells may be washed, removing cell debris and/or accumulated byproducts or the like. In various embodiments bioproduct is separated from the discharge fraction 16, in particular if the bioproduct was secreted into the cell broth 7. The bioproduct may be separated directly by the centrifuge during the centrifugation which produces the centrifuged discharge fraction 16 or may be contained in the supernatant 14 and separated in a later or earlier step. The centrifugation in particular allows changing the culture environment conditions between the source receptacle 2 and the harvest receptacle 3 as supernatant 14 is removed and can be substituted by fresh cultivation medium 8 with different properties. Removing accumulated byproducts further increases the efficiency of the following bioproduction.

At least part of the centrifuged discharge fraction 19, in various embodiments, at least 95 %, at least 99 %, or 100%, of the centrifuged discharge fraction 19, can be combined in step i) with fresh cultivation medium 8 and then used in step ii) to produce bioproduct. In various embodiments, the cells are combined with the fresh cultivation medium 8 during the centrifugation process and this centrifuged discharge fraction is then transferred to the harvest receptacle 3. The fresh cultivation medium 8 may have a volume of less than 90% of the volume of the discharge fraction 16, in various embodiments less than 70%, or less than 50% of the volume of the discharge fraction 16. Here it can be that the cultivation medium 8 of the centrifuged discharge fraction 19 differs in at least one type and/or amount of nutrient from the cultivation medium used in step a) and/or the feed medium used in step b).

In various embodiments, the harvest culture environment conditions in the harvest receptacle 3 are substantially different from the source culture environment conditions. The former may be optimized for bioproduct production and/or the latter for cell growth. Exemplarily, a feed medium 6 may be supplied via a harvest receptacle inlet port 9. The feed medium 6 and/or the feeding profile may be the same feed medium 6 and/or feeding profile as may be used in step b) in the source receptacle 2. However, it can be that the feed medium 6 and/or the feeding profile differs in at least one parameter from the feed medium 6 and/or the feeding profile used in step b) in the source receptacle 2. An example for such a parameter may be the nutrient composition of the feed medium 6.

As depicted in FIG. 1, steps i) and ii) are repeated too, however in various embodiments with different, in particular single-use, harvest receptacles 3 or within a harvest receptacle 3 cleaned between the executions. The source receptacle 2 and the harvest receptacle 3 are not the same receptacle.

In various embodiments, the bioproduct is extracted from a cell broth 7 produced in the harvest receptacle 3 after each execution of step ii). As there can be no further use of the cells from the harvest receptacle 3, the harvest culture environment conditions may be such that the cells die during the bioproduction decreasing the cell viability to a lower value compared to the cultivation in the source receptacle 2.

The duration of step b) and/or step ii) may be at least one day, or at least two days.

In various embodiments, the source receptacle 2 and the harvest receptacle 3 are not directly connected process-wise. In various embodiments, no cells are transferred from the harvest receptacle 3 to the source receptacle 2.

Looking again at the centrifuge 12, the centrifuge 12 can be operated in a forward operation for cell separation and/or cell washing. “Forward operation” means one out of two possible fluid flow directions of a centrifuge 12 and describes the operation leading to a separation of liquid and solid particles, such as culture medium and cells. The liquid obtained this way is the supernatant 14. This separation allows, on the one hand, a washing of separated cells with a washing buffer, such as PBS buffer, or media, such as cultivation medium 8, and/or, on the other hand, to obtain the supernatant 14.

Alternatively, the centrifuge 12 can be operated in a backward operation. “Backward operation” means the second out of two possible fluid flow directions of a centrifuge 12 and describes the operation leading to a discharge of the separated solid particles, such as cells. The product to be obtained in backward operation can be the centrifuged discharge fraction 19.

According to one embodiment it is proposed, that for at least one, at least two, or each execution of steps b) and ii), the cultivation of cells in step b) and the bioproduction in step ii) are executed at least partially in parallel, in various embodiments, that the executions of steps b) and ii) run for at least 50%, at least 75%, or at least 90%, of the time used for cultivation in step b) and/or the time used for bioproduction in step ii) and/or the time used for execution of step b) and/or step ii) in parallel. The times mentioned are the times from the initial filling of the respective receptacle or, partial, re-filling with fresh cultivation medium 8 until the removal of the discharge fraction 16 or removal of at least an essential part of the cell broth 7 of the harvest receptacle 3 for harvesting the bioproduct. If the bioproduct is harvested from the harvest receptacle 3 and/or the discharge fraction 16 is discharged in more than one fraction, the last fraction counts.

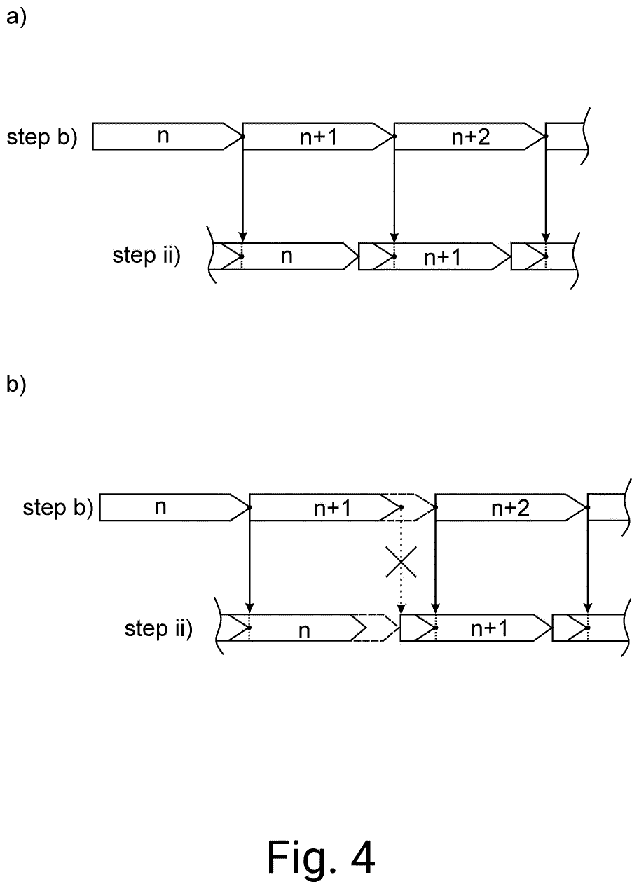

As depicted in FIG. 4a, after step b) was carried out for example for the first time (n=1) as part of the proposed method, a restart fraction 17 of cells may be combined with fresh cultivation medium 8 (step d)) and step b) may be repeated at least a second time (n+1). However, it can be that step b) is repeated until the cyclical production mode is stopped by executing step f) of the proposed method.

As soon as steps c) and i) have been carried out on the discharge fraction 16 of step b) for the first time (n), a first execution of step ii) (n) may be carried out in the harvest receptacle 3. By performing the second execution of step b) (n+1) in parallel to the first execution of step ii) (n), both receptacles operate in parallel. By operating the cultivation in the receptacles in parallel for a predefined amount of time, the utilization of the receptacles and the associated equipment is maximized.

As will be further described below, different strategies may be applied to synchronize the cultivation times in both receptacles.

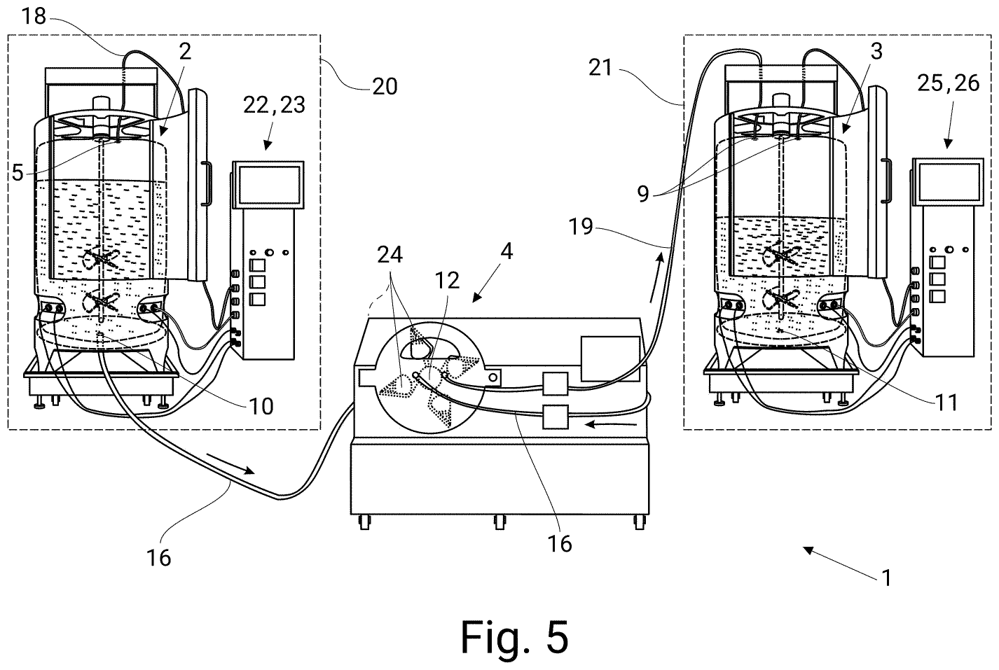

With view towards FIG. 5, a bioproduction may be optimized with regard to general process planning by providing slots for the receptacles, planning movement of media and process times and the like. Therefore, it may be the case, that a single source receptacle slot 20 is paired with a single harvest receptacle slot 21, that step b) is executed for a, in particular pre-determined, cultivation time, that step ii) is executed for a, in particular pre-determined, production time, that the cultivation time and the production time and therefore the source receptacle slot 20 and the harvest receptacle slot 21 are synchronized such that for at least two consecutive executions, such as all executions, of steps ii) the same harvest receptacle slot 21 is used.

One slot comprises exactly one source or harvest receptacle 3, such as at least or exactly one electronic process control for the receptacle and a defined location.

In various embodiments, after at least one repetition, in some embodiments after each repetition, of steps b), c) and i), the harvest receptacle slot 21 is already prepared to receive the centrifuged discharge fraction 19. When the term “repetition” is used, any execution except the first execution is meant. Therefore, the method is synchronized such that the harvest receptacle 3 is not only finished, but also necessary steps for preparation of the harvest receptacle slot 21, for example connecting a new receptacle, are done. In various embodiments, the harvest receptacle slot 21 is already prepared when the first fraction of the centrifuged discharge fraction 19 is ready to be discharged from the centrifuge 12 or at least it is prepared so soon that no relevant part of the cells die.

The term “pairing” presently means that the cells cultivated in the source receptacle 2 of the source receptacle slot 20 are transferred to the harvest receptacle 3 located in the harvest receptacle slot 21. The pairing of a source receptacle slot 20 and a harvest receptacle slot 21 can be maintained for at least 2 executions of steps b) and ii) and/or, in some embodiments, for at least 3 weeks, or at least 5 weeks.

As depicted in FIG. 5, the source receptacle slot 20 comprises the physical space to receive the source receptacle 2. In various embodiments, the source receptacle slot 20 comprises the physical place to install the source receptacle 2. As depicted in FIG. 5, transport means may be used to transport the source receptacle 2 to the space provided by the source receptacle slot 20. It can be that the source receptacle slot 20 comprises a source electronic process control 22 and that the source electronic process control 22 is integrated into a source electronic process control unit 23. In various embodiments, the source electronic process control unit 23 is located next to the source receptacle 2.

In various embodiments, the source electronic process control unit 23 is connected to at least one sensor placed inside or at the source receptacle 2. As depicted in FIG. 5, this sensor might be a pH probe or a temperature probe or any other sensor suited to monitor and preferably also control the at least one process parameter.

In various embodiments, cultivation in the source receptacle slot 20 is carried out for a pre-determined cultivation time. After the predetermined cultivation time, a discharge fraction 16 of the cell broth 7 is discharged from the source receptacle 2 and transferred to the centrifuge 12.

As further depicted in FIG. 5, the source receptacle 2 is connected to the clarification setup 4 comprising the centrifuge 12. Here it is to be noted that the complex network of fluid lines that guides the liquids to and from the centrifuge chambers 24 is not depicted to maintain clarity. In various embodiments, the centrifuge 12 is designed as a fluidized-bed centrifuge 12 comprising at least one centrifuge chamber 24. In various embodiments and as depicted in FIG. 5, the centrifuge 12 comprises four centrifuge chambers 24.

It is to be noted here, that the connection between the source receptacle 2 and the centrifuge 12 and/or the harvest receptacle 3 and the centrifuge 12 is, in some embodiments, only maintained until the respective transfer steps have been carried out. This way, the centrifuge 12 may be utilized elsewhere, when no transfer to and/or from the centrifuge 12 and no centrifugation is required.

As depicted in FIG. 5, the harvest receptacle slot 21 comprises the physical space to receive the harvest receptacle 3. In various embodiments, the harvest receptacle slot 21 comprises the physical place to install the harvest receptacle 3. As depicted in FIG. 5, transport means may be used to transport the harvest receptacle 3 to the space provided by the harvest receptacle slot 21. It can be that the harvest receptacle slot 21 comprises a harvest electronic process control 25 and that the harvest electronic process control 25 is integrated into a harvest electronic process control unit 26. In various embodiments, the harvest electronic process control unit 26 is located next to the harvest receptacle 3.

In various embodiments, the harvest electronic process control unit 26 is connected to at least one sensor placed inside or at the harvest receptacle 3. As depicted in FIG. 5, this sensor might be a pH probe or a temperature probe or any other sensor suited to monitor and, in various embodiments, also control the at least one process parameter.

As becomes clear from FIG. 5, in various embodiments, the source receptacle 2 and the harvest receptacle 3 are not directly connected. Further, and as also depicted in FIG. 5, once the cell broth 7 has been transferred from the source receptacle 2 to the centrifuge 12, the cell broth 7 is not transferred back to the source receptacle 2. Generally it would be possible to submit the whole cell broth 7 from the source receptacle 2 to the centrifuge 12 and transfer the restart fraction 17 from the centrifuge, directly, or indirectly for example via the harvest receptacle 3, back to the same and not cleaned, sterilized or the like source receptacle 2. In various embodiments, however, the restart fraction 17 stays inside the source receptacle 2.

In various embodiments steps a) to f) are synchronized, time-wise, with repeated seed trains such that after step f) step a) is repeated at the same source receptacle slot 20 without breaking synchronicity between the source receptacle slot 20 and the harvest receptacle slot 21. After a number of repetitions of step b), the cells may become genetically unstable. In various embodiments, they are replaced with new initial cells 15 from a seed train without changing the repetitions in the harvest receptacle 3. In that way, production can be maintained forever in theory.

According to one embodiment it is proposed, that the method further comprises the step of iii) transferring a harvest fraction 27 from the harvest receptacle 3 to the clarification setup 4 and centrifuging the harvest fraction 27 via the centrifuge 12, thereby separating the bioproduct, when located in the supernatant 14 from the harvest fraction 27. In various embodiments, step iii) is executed after each step ii). In various embodiments, the harvest fraction 27 is the complete cell broth 7 from the harvest receptacle 3. Again, the bioproduct, when located in the supernatant 14, may be forwarded to further purification steps 29 while the harvest fraction 28 can be directed to a waste 13 location. Alternatively, when the bioproduct is present within the cells, the harvest fraction 28 may be directed to further purification steps and the supernatant may be directed to a waste location.

In various embodiments the same clarification setup 4 is used in step i) and step iii).

According to one embodiment it is proposed, that the bioprocess installation 1 comprises a sensor arrangement, that the sensor arrangement comprises at least one sensor placed in or at the source receptacle 2 and/or at least one sensor placed in or at the harvest receptacle 3, in various embodiments, that at least one sensor is configured to measure at least one process parameter out of the group of oxygen concentration, carbon dioxide concentration, pH, temperature, conductivity, pressure, viable cell concentration, viable cell production rate, product concentration, productivity, stirring speed and/or the culture medium, including the concentration of nutrients like carbon-source concentration, nitrogen-source concentration, amino acid concentration, growth factor concentration or the like for the source receptacle 2 and/or for the harvest receptacle 3. As mentioned, the sensor may be connected to the respective electronic process control unit and used to control the respective culture environment conditions in the source receptacle 2 and/or in the harvest receptacle 3.

The term “process parameter” is to be understood in a broad sense and includes at least one process parameter that may be monitored and, in various embodiments, also controlled within the source receptacle 2 and/or the harvest receptacle 3.

It should be noted that additional process parameters may be derived from monitoring more than one process parameter. For example, the change in the bioproduct concentration over time, which may also be referred to as productivity, may be derived by relating the bioproduct concentration to the cultivation time. Further, the cell growth rate may be determined from evaluating the change of the viable cell concentration over the cultivation time.

As already mentioned, in may be the case, that the bioprocess installation 1, in particular the source receptacle slot 20, comprises a source electronic process control 22 for controlling the source culture environment conditions, in various embodiments, that the source electronic process control 22 is connected to the at least one sensor placed in or at the source receptacle 2.

Further it may be the case, that step ii) is terminated after a pre-determined termination condition is reached, in various embodiments, that at least one sensor measurement related to the termination condition is measured repeatedly, in particular cyclically or continuously, by the sensor placed in or at the harvest receptacle 3 during at least one, in particular each, execution of step ii), in some embodiments, that the sensor measurement is used, in particular by a harvest electronic process control 25, to predict a time of reaching the termination condition, that the source culture environment conditions in step c) are controlled, in particular by the source electronic process control 22, to synchronize step c) with step ii). The sensors or measured process parameters named above apply.

What's very interesting now is that in some cases, the harvest receptacle 3 may be used for longer or shorter than planned and that may be noticed or deliberately decided only during the respective step ii). Instead of having to interrupt step ii) because step b) is about to finish, step b) may be planned with a control reserve from the beginning. In various embodiments, step b) can be slowed down or sped up. This leads to longer than the theoretical best for the duration of step b), but this change may in particular be used to keep the viability of the cells in the source receptacle 2 above a pre-defined threshold and/or to produce more bioproduct already during step b) and gaining a control reserve to speed up step b). Additionally or alternatively, step b) may be slowed down by changing the source culture environment conditions to produce more bioproduct and/or have less cell growth and thereby for example less toxic byproduct production, which may decrease the overall culture viability.

In general, a synchronization strategy may be executed, in particular applied to the source receptacle 2, to keep the source and harvest receptacles 3 and/or their slots synchronized. Such a synchronization is particularly important, as the cells may not be kept viable within the source receptacle 2 for a prolonged time without adjusting the source culture environment conditions and/or at least one process parameter within the source receptacle 2. The goal is to provide a pre-defined amount of cells in the source receptacle 2 when the harvest receptacle slot 21 has been prepared to execute step ii). For this, the cultivation time in the source receptacle 2 is synchronized with the cultivation time in the harvest receptacle 3.

Such a synchronization strategy is depicted in FIG. 4b. For example, a temperature inside the source receptacle 2 may be reduced to prolong the cultivation time in the source receptacle 2. Additionally or alternatively, feed medium may be supplied to the cultivated cells or the supply of feed medium may be continued to prolong the cultivation time in the source receptacle 2.

The respective process parameters may be predicted based on historical data. In case the prediction is not good enough, incremental changes to the process parameters may be made. Another synchronization strategy may include a change in the split of the cell broth 7 from the source receptacle 2 into discharge fraction 16 and restart fraction 17. If more cell broth 7 or a higher viable cell concentration has been reached in a prolonged step b), the restart fraction 17 may be decreased, either absolutely or relatively, to allow another longer step b) and/or step ii).

A pre-determined termination condition for the harvest receptacle 3 may be that at least one process value reaches a certain value, in particular, and as indicated in FIG. 3, that the viability (viab) falls below a predetermined threshold, and/or, that a certain cultivation time is reached, and/or, that a certain filling volume is reached, and/or, that, a certain bioproduct concentration is reached, and/or, that a ratio between product and by-product concentration falls below a predetermined threshold, and/or, that a certain amount of byproduct is reached, and/or, that the viable cell concentration reaches a certain value, and/or, that a certain process value was maintained for a certain amount of time.

In order to estimate the time, when the cultivation in the harvest receptacle 3 will reach the pre-defined termination condition, various process parameters with appropriate process models can be used, such as cell viability, cell specific productivity, product concentration, level of impurities (e.g. host cell impurity concentration) or any CQA of the product.

According to one embodiment it is proposed, that if a deviation between the predicted time of reaching the termination condition and a planned end of the production time is detected, in particular by the source electronic process control 22 or the harvest electronic process control 25, a synchronization strategy is executed, in various embodiments, that the synchronization strategy comprises adapting the source culture environment conditions and/or harvest culture environment conditions to reduce the resulting deviation between the source receptacle 2 and the harvest receptacle 3, in particular to reach a pre-defined absolute cell quantity in the source receptacle 2 prior to initiating step c). This may lead to step c) starting earlier or later.

Another synchronization strategy may include changing a composition of the feed medium 6 and/or a point in time of adding the feed medium 6 and/or changing a target filling volume in the source receptacle 2.

According to one embodiment it is proposed, that an initial viable cell concentration in step ii) is higher than a final viable cell concentration in step b), in various embodiments, that the cell concentration is increased by the centrifugation in step i). This may be particularly advantageous also in a case where the source receptacle 2 has a bigger volume than the harvest receptacle 3, for example at least 10% bigger.

It may also be the case, that at least one process parameter of the source culture environment conditions, in particular initial source culture environment conditions, in at least one, in particular each, execution of step b) and of the harvest culture environment conditions, in particular initial harvest culture environment conditions, in at least one, in particular each, execution of step ii) is set differently, in various embodiments, that the at least one process parameter is optimized in the source culture environment conditions for cultivation of cells over production of bioproduct and/or that the at least one process parameter is optimized in the harvest culture environment conditions for production of bioproduct over cultivation of cells. Different here means substantially different, not different within tolerances or near tolerances.

According to one embodiment it is proposed, that the initial source culture environment, in particular the source culture environment, and/or the initial harvest culture environment, in particular the harvest culture environment, is essentially identical for at least one, in particular each, repetition of step b) and/or step ii) respectively.

According to one embodiment it is proposed, that cultivating the cells in step b) of the cyclical production mode is performed under batch cultivation conditions or fed-batch cultivation conditions, and/or, that the production mode of step ii) is performed under fed-batch cultivation conditions.

This may be the case at least once, at least twice, or each time the respective step is executed. According to one embodiment and as already mentioned it is proposed, that the centrifuge 12 is a fluidized bed centrifuge 12, in various embodiments, that the centrifuge 12 is operated in a forward operation for cell separation and/or cell washing and a backward operation for cell discharging, in various embodiments, that the centrifuge 12 is operated in a backward operation for transferring the centrifuged discharge fraction 19 to the harvest receptacle 3 between steps i) and ii).

According to one embodiment it is proposed, that a transfection step is carried out after step c) on the discharge fraction 16, or, that the bioproduct is produced by the cells in the source receptacle 2.

Here, the use of a centrifuge 12, such as a fluidized-bed centrifuge, as the clarification setup 4 provides simple means to carry out a transfection step. For the definition of transfection, reference is made to the definition given above. For example, by being able to supply a wash solution and/or fresh culture medium during centrifugation in step i), treatment with calcium phosphate for transfection may easily be carried out as the DNA to be transferred into the cells and the calcium phosphate may be supplied during the centrifugation. Transfection may also be carried out prior to or after centrifugation outside the source receptacle 2.

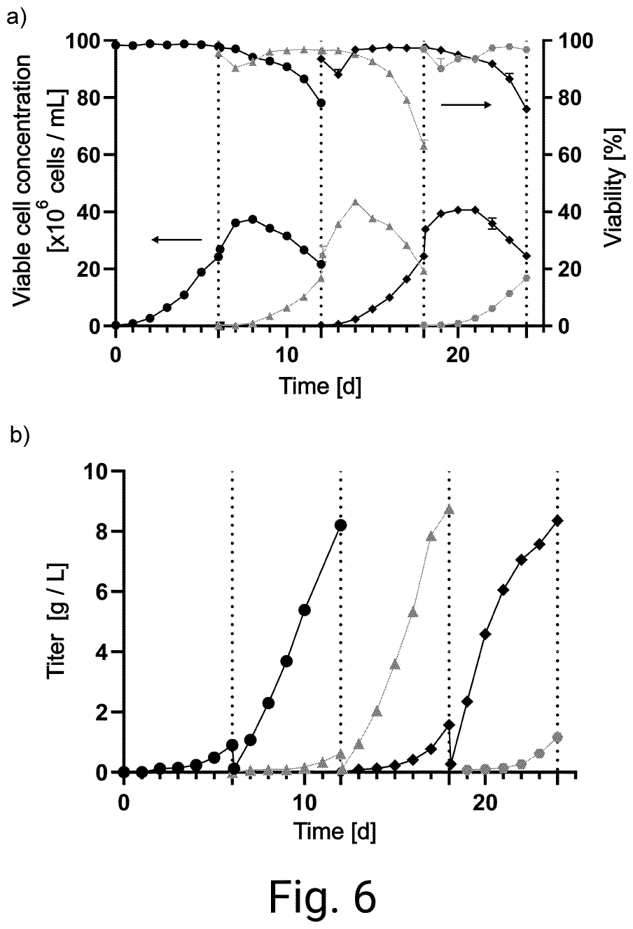

The results for proof-of-concept experiments are shown in FIG. 6. Here, two 5 L glass Univessel were used as source receptacle 2 and harvest receptacle 3, respectively. Cells were cultivated in the source receptacle 2 for six days until a viable cell concentration (VCC) of about 25×10\hat{ }6 cells per milliliter was reached (FIG. 6 a), full circles). At this point, indicated by the dashed vertical line in FIG. 6a, a discharge fraction 16 of the cell broth 7 was discharged from the source receptacle 2 to the harvest receptacle 3 and the cells were cultivated for another six days in the harvest receptacle 3 (FIG. 6 a), full circles). At the same time, a restart fraction 17 of the cell broth 7 was combined with fresh cultivation medium 8 in the source receptacle 2 such that the initial viable cell concentration (VCC) was 0.3×10\hat{ }6 cells per mL (FIG. 6 a), shaded triangles). This process was repeated several times as indicated in FIG. 6 a) (diamonds and shaded circles). The dashed vertical lines indicate a change of receptacle.

The results demonstrate that operating the source receptacle 2 in a cyclical production mode enables the cyclical production of viable cells and that cell growth and viability are not negatively affected by the cyclical production mode.

As further indicated in FIG. 6, the results demonstrate that a non-cyclical operation of the harvest receptacle 3 enables adjusting the process conditions in the harvest receptacle 3 to favor bioproduction (FIG. 6b) without having to keep the cells viable (FIG. 6a). While the cell viability decreases during the operation of the harvest receptacle 3 in production mode (FIG. 6a), the concentration of the bioproduct, in this case an antibody, increases disproportionately. Overall, the proposed method reveals not only similar performance compared to previous processes but as well shows a continuous characteristic further enhancing process efficiency.

### REFERENCE NUMBERS

1 bioprocess installation
2 source receptacle
3 harvest receptacle
4 clarification setup
5 source receptacle inlet port
6 feed medium
7 cell broth
8 cultivation medium
9 harvest receptacle inlet port
10 source receptacle outlet port
11 harvest receptacle outlet port
12 centrifuge
13 waste
14 supernatant
15 initial cells
16 discharge fraction
17 restart fraction
18 feed line
19 centrifuged discharge fraction
20 source receptacle slot
21 harvest receptacle slot
22 source electronic process control
23 source electronic process control unit
24 centrifuge chamber
25 harvest electronic process control
26 harvest electronic process control unit
27 harvest fraction
28 centrifuged harvest fraction
29 purification step

1. A method for operating a bioprocess installation for production of a bioproduct, wherein the bioprocess installation comprises a source receptacle for cell cultivation, a harvest receptacle for bioproduction and a clarification setup with a centrifuge,
wherein the source receptacle is operated in a cyclical production mode,
wherein the cyclical production mode comprises, in this order, the steps of:
a) starting the cyclical production mode in the source receptacle with initial cells and cultivation medium,
b) cultivating the cells in the source receptacle, thereby obtaining a cell broth comprising cultivated cells, whereby the source receptacle and the cultivation medium are configured to provide source culture environment conditions for the cultivation of the cells,
c) discharging a discharge fraction of the cell broth from the source receptacle,
d) combining a restart fraction of the cell broth with fresh cultivation medium and repeating step b),
e) repeating steps c) and d) at least once and/or
f) discharging the cell broth obtained from step d) from the source receptacle stopping the cyclical production mode, obtaining a discharge fraction,
wherein the method further comprises the steps of:
i) transferring the discharge fraction to the clarification setup and centrifuging the discharge fraction via the centrifuge thereby separating the discharge fraction into at least a centrifuged discharge fraction and supernatant,
ii) transferring at least part of the centrifuged discharge fraction from step i) and fresh cultivation medium into the harvest receptacle and operating the harvest receptacle in a production mode, whereby the harvest receptacle and the cultivation medium are configured to provide harvest culture environment conditions, producing the bioproduct in the harvest receptacle,
wherein steps i) and ii) are executed at least twice with discharge fractions from an execution of step c) and/or step f).

2. The method according to claim 1, wherein for at least one execution of steps b) and ii), the cultivation of cells in step b) and the bioproduction in step ii) are executed at least partially in parallel.

3. The method according to claim 1, wherein a single source receptacle slot is paired with a single harvest receptacle slot, that step b) is executed for a cultivation time, that step ii) is executed for a production time, that the cultivation time and the production time and therefore the source receptacle slot and the harvest receptacle slot are synchronized such that for at least two consecutive executions of steps ii) the same harvest receptacle slot is used.

4. The method according to claim 3, wherein steps a) to f) are synchronized with repeated seed trains such that after step f) step a) is repeated at the same source receptacle slot without breaking synchronicity between the source receptacle slot and the harvest receptacle slot.

5. The method according to claim 1, wherein the method further comprises the step of
iii) transferring a harvest fraction from the harvest receptacle to the clarification setup and centrifuging the harvest fraction via the centrifuge thereby separating the bioproduct from the harvest fraction.

6. The method according to claim 1, wherein the bioprocess installation comprises a sensor arrangement, that the sensor arrangement comprises at least one sensor placed in or at the source receptacle and/or at least one sensor placed in or at the harvest receptacle.

7. The method according to claim 1, wherein the bioprocess installation comprises a source electronic process control for controlling the source culture environment conditions.

8. The method according to claim 1, wherein step ii) is terminated after a pre-determined termination condition is reached.

9. The method according to claim 3, wherein if a deviation between the predicted time of reaching the termination condition and a planned end of the production time is detected, a synchronization strategy is executed.

10. The method according to claim 1, wherein an initial viable cell concentration in step ii) is higher than a final viable cell concentration in step b).

11. The method according to claim 1, wherein at least one process parameter of the source culture environment conditions, in at least one execution of step b) and of the harvest culture environment conditions, in at least one execution of step ii) is set differently.

12. The method according to claim 1, wherein the initial source culture environment and/or the initial harvest culture environment is essentially identical for at least one repetition of step b) and/or step ii) respectively.

13. The method according to claim 1, wherein cultivating the cells in step b) of the cyclical production mode is performed under batch cultivation conditions or fed-batch cultivation conditions, and/or, that the production mode of step ii) is performed under fed-batch cultivation conditions.

14. The method according to claim 1, wherein the centrifuge is a fluidized bed centrifuge.

15. The method according to claim 1, wherein a transfection step is carried out after step c) on the discharge fraction, or, that the bioproduct is produced by the cells in the source receptacle.

16. The method according to claim 2, wherein the executions of steps b) and ii) run for at least 50% of the time used for cultivation in step b) and/or the time used for bioproduction in step ii) and/or the time used for execution of step b) and/or step ii) in parallel.

17. The method according to claim 5, wherein step iii) is executed after each step ii).

18. The method according to claim 6, wherein at least one sensor is configured to measure at least one process parameter out of the group of carbon-source concentration, nitrogen-source concentration, amino acid concentration, growth factor concentration, oxygen concentration, carbon dioxide concentration, pH, temperature, conductivity, pressure, biomass concentration, biomass production rate, product concentration, productivity, oxygen uptake rate and/or stirring speed for the source receptacle and/or for the harvest receptacle.

19. The method according to claim 7, wherein the source electronic process control is connected to the at least one sensor placed in or at the source receptacle.

20. The method according to claim 1, wherein step ii) is terminated after a pre-determined termination condition is reached, wherein at least one sensor measurement related to the termination condition is measured repeatedly, cyclically or continuously, by the sensor placed in or at the harvest receptacle during at least one execution of step ii), wherein the sensor measurement is used by a harvest electronic process control, to predict a time of reaching the termination condition, that the source culture environment conditions in step b) are controlled, by the source electronic process control, to synchronize step b) with step ii).
